(19) United States
(12) Patent Application Publication
(10) Pub. No.: US 2001/0041675 A1
(43) Pub. Date: Nov. 15, 2001

(54) Satisfy composition and method of weight control

(76) Inventor: Robert H. Jacobs, Marina Del Rey, CA (US)

Correspondence Address: Ronald J. Clark, P.O. Box 8539, Newport Beach, CA, 92658, US

(21) Appl. No.: 09/766,989

(22) Filed: Jan. 22, 2001

Related U.S. Application Data
(60) Provisional application No. 60/177,386, filed on Jan. 24, 2000.

(51) Int. Cl. A61K 31/70; A61K 31/555; A61K 33/26; A61K 35/78

(52) U.S. Cl. 514/23; 424/646; 424/769; 424/728; 514/184

(57) ABSTRACT

The present invention comprises a number of nutritional supplements, or agents and/or formulas. A first nutritional formula is designed for eliminating the cravings for consuming calories, for regulating appetite, and for countering the feeling(s) of weakness and tiredness that may encourage and or drive a person to eat when their body does not require nutritional sustenance. This first nutritional supplement is formulated to be used alone and/or selectively combined with a second nutritional agent which is a thermogenic formulated fat burning agent. This second thermogenic agent is designed to enhance fat burning. The second thermogenic agent can also be used alone. A third nutritional agent is formulated for minimizing the tendency to store fat. The third nutritional agent can also be used alone. Its effect is enhanced by use with either one or both of the other two agents.

### CROSS REFERENCE TO RELATED APPLICATIONS

Provisional patent applications No. 60/177,386, filed Jan. 24, 2000 and entitled “Satisfy a stimulant for the hypothalamus.”

### STATEMENT REGARDING FEDERALLY SPONSORED RESEARCH OR DEVELOPMENT

Not Applicable

### REFERENCE TO A “MICROFICHE APPENDIX”

Not Applicable

### BACKGROUND OF THE INVENTION

Field of the Invention

This Invention relates to one or more of a combination of weight reduction nutritional agents used alone or in combination with any one and/or other(s) of the described nutritional agents or formulas.

### BRIEF SUMMARY OF THE INVENTION

The Satisfy weight management system including the present invention comprises a number of nutritional supplements, or agents and/or formulas. One of present inventions of this patent is a first nutritional formula for the ingredient called Satisfy #1. This first nutritional supplement is designed for eliminating the cravings for consuming calories, for regulating appetite, and for countering the feeling(s) of weakness and tiredness that may encourage and or drive a person to eat when their body does not require nutritional sustenance. This first nutritional supplement is formulated to be used alone and/or selectively combined with a second nutritional agent which is a thermogenic formulated fat burning agent. This second thermogenic agent is designed to enhance fat burning, and is called Satisfy #2. The second thermogenic agent can also be used alone. A third nutritional agent is formulated for minimizing the tendency to store fat. This is a fiber supplement called Satisfy #3. The third nutritional agent can also be used alone. Its effect is enhanced by use with either one or both of the other two agents. These three agents, plus exercise, provide a comprehensive program for successful weight management.

### BACKGROUND ART

None is known at this time.

### BRIEF DESCRIPTION OF THE SEVERAL VIEWS OF THE DRAWINGS

There are no drawings in this application.

### BRIEF SUMMARY OF THE INVENTION

In one aspect of the invention, the first nutritional agent is formulated to moderate the cravings that cause a person to overeat, or to snack on unhelpful high calorie foods that promote weight gain, These cravings originate in the hypothalamus. The hypothalamus is the regulatory center of the brain that controls blood sugar, feelings of satiety and hunger, and internal homeostasis. When the hypothalamus becomes upset due to a drop in brain blood sugar levels, it gives the order to the body to replenish blood sugar supplies. Since blood sugar is the brain's fuel, appropriate levels are critical to brain function and the brain is ultra sensitive to even minimal fluctuations. When the blood sugar level(s) drop too low, the hypothalamus becomes disturbed and tells the brain to seek sugar. This in turn creates the urge to consume calories in general or to eat in particular. As a result of low brain blood sugar level(s), a person may become agitated, disturbed, tired or hungry and reach for food. Often they may over-supply the brain's need for a little blood sugar, by taking in an abundance of unnecessary calories that contribute to excess weight and stress the body.

The first nutritional agent, called Satisfy #1, of the invention rectifies the brain's blood sugar imbalance quickly and effectively. This reduces the need for an excessive intake of additional calories. This aspect of the invention consists of a glucose wafer, which contains the blood sugar stabilizing minerals of vanadium and chromium. The wafer is dissolved in the mouth and the glucose passes directly under the tongue into the blood stream. Within seconds the brain receives an influx of blood sugar. As a result, the hypothalamus becomes satisfied and sends a signal of satiety to the rest of the body. Using the first nutritional agent alone, a person is no longer driven by cravings to overeat and can more easily manage their diet to achieve their weight-loss goals.

Until now the hypothalamus has never been directly addressed for switching off such cravings. Dieting solutions have approached moderating such cravings chemically, with such factors as using phenylpropanolamine, which is a drug to reduce appetite, or with amphetamines, which create a change in neuro transmitter levels and offset appetite. Other proposed solutions have involved elevating seratonin levels to try to quiet the hypothalamus. Although such approaches have been of some use in a round about way, the hypothalamus has never simply and directly been given what its asking for which is brain blood sugar. This is the first time direct application of blood sugar to the brain has ever been used to satisfy appetite.

The following is a description of the dosage for the first nutritional agent called Satisfy #1 . Each Satisfy #1 wafer consists of two grams of USP grade glucose in the form of dextrose, which supplies about 7 to 10 calories of energy to the body. The dose is determined by ½ of the amount that a diabetic would need in a hypoglycemic episode. In such instances, generally 4 grams are given to bring the blood sugar up to a normal level. This weight management program is not recommended for diabetic or severely hypoglycemic patients. The selected dosage is low enough to rapidly increase blood sugar levels to satisfy the hypothalamus, without destabilizing overall blood sugar balance.

The optimal dosage range for glucose is somewhere between ½-4 grams, depending upon a person's weight and individual biochemistry. An individual's “magic point” at which their hypothalamus becomes “happy” will vary considerably. For one person a single wafer will suffice to switch off cravings for a matter of hours. Another person might need two or more wafers at more regular intervals. In addition a person's cravings and need for Satisfy #1 will fluctuate according to how much stress they are under.

The wafers also contain vanadium sulfate and chromium polynicotinate, which help regulate blood sugar levels. These minerals specifically improve the body's long-term ability to sustain healthy blood sugar levels. Each wafer preferably contains about 50 mcg of chromium polynicotinate and 10 mcg of vanadium sulfate. The optimal range of chromium polynicotinate per day would be between 150-400 mcg, not exceeding 1000 mcg. The optimal range of vanadium sulfate would be between 15-40 mcg per day, not exceeding 100 mcg.

Applications for the Effective Use of Satisfy #1 Wafers to promote Weight Loss.

1. For appetite control

A Satisfy wafer may be taken prior to a meal in order to take the edge off extreme hunger pangs. By supplying immediate blood sugar to the hypothalamus at the start of a meal a certain degree of satiety is reached which discourages a person from eating more than their body may truly want or need.

2. To control cravings between meals

Satisfy wafers may be taken between meals in place of snacks to override cravings that might otherwise lead to the consumption of high quantities of calories.

3. To balance emotional states of stress and distress

The Satisfy wafers can also be used to bring satiety to the hypothalamus when a person is experiencing emotional distress that might lead to bingeing.

The wafers are recommend to be used in conjunction with the Weight Management Plan Booklet, which covers such issues as balancing blood sugar and insulin levels, enhancing fat burning, controlling calorie intake, increasing metabolic rate through exercise, as well as meal suggestions and tips.

A second thermogenic nutritional agent, called Satisfy #2, is employed for encouraging the body to burn fat. One or more new and improved thermogenic formulas are provided. The user is suggested to select one of the formulas for use. The first formula for the second nutritional agent called Satisfy #2, helps encourage weight loss by increasing the body's ability to burn fat through a heat-generating process known as thermogenesis. Its two main fat-burning ingredients are caffeine and ephedrine in combination. The first formula for the second nutritional agent called Satisfy #2, formula contains the natural sources of caffeine, in the form of guarana and ekola nut, and ephedrine in the form of Ma Huang and Kava Kava. These plant sources also provide a range of cofactors, including various alkaloids, that improve the activity of the chemicals in the body. The Satisfy #2 formula also includes fat mobilizers and adrenal stabilizers. The adaptogen Siberian ginseng supports adrenal balance. It helps to regulate blood sugar levels and promote sustained energy levels to prevent feelings of weakness and fatigue. The amino acid L-Carnitine helps mobilize fat and make fat stores available to be burned for energy. White willow bark, which contains salicylic acid, works synergistically with ephedrine and caffeine to encourage thermogenesis. Iodine in the formula, extracted from sea vegetables, acts as a thyroid moderator. Satisfy #2 also includes Vitamin B6, as an important enzyme co-factor, and chromium to help balance blood sugar levels.

One of the problems with other thermogenic formulas is that the stimulant ephedrine and caffeine can provoke the unwanted side effects of nervousness and irritability. Indeed many thermogenic formulas may be dangerously stimulating and can cause such symptoms as heart palpitations, jitters, sweating and ultimately exhaustion. Such reactions are minimized in the first formula for the second nutritional agent called Satisfy #2, by the addition of Kava kava root, which acts as a calming agent. Kava kava neutralizes the stimulating effects of ephedrine and caffeine—allowing a person to achieve the thermogenic effect without the unwanted side effects.

A second formula for the second nutritional agent called Satisfy #2 replaces Kava Kava and Ma Huang with DL-Phenylalanine. The DL-Phenylalanine is converted into norepinephrine, dopamine, and epinephrine, which are neurotransmitters. These substances dramatically affect energy, hunger, and mood in a positive way. DL-Phenylalanine also extends the life of endorphins in the brain, which gives the person a feel good effect. DL-Phenylalanine also is converted into phenylethylamine, which is the mood enhancer found in chocolate.

Dosages of main ingredients in the first formula for the second nutritional agent called Satisfy #2 include 100 mgs of guarana, rated at 12% caffeine. An alternate source for caffeine is green tea. 150 mgs of Ma Huang rated at 8% ephedrine, 100 mgs of L-Carnitine, 50 mcg of chromium polynicotinate, 50 mg of kava kava, 10 mgs of Vitamin B6, 100 mcg of iodine. Dosages of the main ingredients in the second formula for the second nutritional agent include 100 mgs of guarana, rated at 12% caffeine or an equal caffeine amount of green tea, 100 mgs of L-Carnitine, 50 mcg of chromium polynicotinate, 10 mgs of Vitamin B6, 100 mcg of iodine, and 87.5 mg of phenylalanine.

The third nutritional agent, called Satisfy #3, is employed for Insulin management. It can be used alone or in combination with either formula of the second nutritional agents, and or with the first nutritional agent.

The biochemical process by which blood sugar is stored as fat is controlled by the hormone insulin. When levels of insulin are high, metabolism slows down and the body tends to store fat more readily. The result is an increase in weight gain. Certain types of digested foods, especially refined sugar and high carbohydrate foods, enter the bloodstream very quickly and subsequently cause a rapid rise in insulin levels. The third nutritional agent, called Satisfy #3, is a fiber supplement and is designed to slow down the rate at which sugars and fats, from digestion, enter the blood stream. Satisfy #3's soluble fiber encapsulates the digested food in a gelatinous matrix, so that its absorption is gradual. The steady stream of sugars and fats into the blood allows lower insulin levels to be maintained. In this way Satisfy #3 helps prevent the storage of excess fat that can result when insulin levels are spiked by a rush of blood sugar. Many weight loss plans attempt to balance insulin with complex and stringent diet plans. Satisfy #3 accomplishes the same insulin management and weight control by just adding fiber capsules to each meal.

The recommended dosage for Satisfy #3 is two 400 mg capsules with each meal. Satisfy #3 comprises a proprietary blend of psyillium husks, guar gum, oat fiber and apple pectin.

The use of all three nutritional agents in Satisfy weight management system is unique because it:

1. Manages cravings without heavy drug/chemical intervention thanks to the first nutritional agent.

2. Increases thermogenic activity safely and without causing nervousness or overstimulation thanks to either formula of the second nutritional agent.

3. Balances insulin levels and offsets unnecessary fat storage thanks to the composition, including fiber in the third nutritional agent.

Although the invention has been described in detail with references to a number of preferred embodiments, it will be understood by one of ordinary skill in the art that various modifications can be made without departing from the spirit and scope of the invention. The proceeding examples can be repeated with similar success by substituting the generically or specifically described reactants and or operation conditions of this invention for those specifically used in the examples. Accordingly, it is not intended that the invention be limited, except by the appended claims.

I claim:

1. A weight control composition for eliminating the cravings to consume excessive calories in a living mammal having a brain which requires a homeostatic brain blood sugar level which requires no additional intake into the living mammal of additional calories, and having an hypothalamus for sensing such homeostatic brain blood sugar level in order to create and send feeling to the living mammal of satiety and or hunger and or internal homeostasis, comprising:
A nutritional agent comprising between ½ to 4 grams of USP grade glucose, in the form of dextrose, for quickly and effectively raising brain blood sugar levels in the brain towards the homeostatic brain blood sugar level, and for causing the hypothalamus to detect such homeostatic brain blood sugar level and thereby satisfying the hypothalamus's regulation of satiety, hunger and internal homeostasis and for reducing the need for an excessive intake of additional calories by the living mammal.

2. The weight control composition as recited in claim 1, and further comprising:
A weight of mineral vanadium sulfate selected from the range of 15-40 mcg and not to exceed 100 mcg per day for providing blood sugar stabilization to the hypothalamus.

3. The weight control composition as recited in claim 1, and further comprising:
A weight of mineral chromium polynicotinate selected from the range of 150 to 400 mcg and not to exceed 1000 mcg per day.

4. The weight control composition as recited in claim 1, and further comprising:
A weight of mineral vanadium sulfate selected from the range of 15-40 mcg and not to exceed 100 mcg per day for providing blood sugar stabilization to the hypothalamus; and
A weight of mineral chromium polynicotinate selected from the range of 150 to 400 mcg and not to exceed 1000 mcg per day.

5. The weight control composition as recited in claim 1, and further comprising:
A natural source of Caffeine selected from one or more of guarana and ekola nut selected from the range of 75 mg to 125 mg, and preferably 100 mg of guarana and from a range of 40 mg, to 70 mg, and preferably 50 mg of ekola nut;
A natural source of ephedrine, rated as 8% ephedrine, in the form of Ma Huang selected from the range of 125 mg to 175 mg and preferably 150 mg;
A natural source of Siberian ginseng selected from the range of 60 mg to 80 mg, and preferably 75 mg as an adrenal stabilizer;
A natural source of amino acid L-Carnitine as a fat mobilizer selected from the range of 80 mg to 120 mg, and preferably 100 mgs;
A natural source of saticylic acid in the form of white willow bark for encouraging thermogenesis selected from the range of 20 mg to 30 mg and preferably 25 mg;
A natural source of iodine in the form such as extracted from sea vegetables as a thyroid moderator selected from the range of 90 mcg to 110 mcg, and preferably 100 mcg;
An amount of Vitamin B6 selected from the range of 7 mg to 15 mg, and preferably 10 mg, for use as an enzyme co-factor;
An amount of chromium polynicotinate selected from the range of 40 mcg to 65 mcg, and preferably 50 mcg, for use in balancing blood sugar levels; and
An amount of Kava Kava for partially neutralizing the stimulating effects of ephedrine and caffeine selected from the range of 45 mg to 55 mg, and preferably 50 mgs.

6. The weight control composition as recited in claim 1, and further comprising:
A weight of DL-Phenylalanine selected from the range of 70 mg to 90 mg and preferably 80 mg for conversion by the living mammal into norepinephrine, dopamine, and epinephrine for use as neurotransmitters, and for use as affecting energy, hunger, and mood in a positive direction, and for extending the life of endorphins in the brain;
Said DL-Phenylalanine being converted by the living mammal into phenylethylamine as a positive mood enhancer for the living mammal;
A natural source of Siberian ginseng selected from the range of 60 mg to 80 mg, and preferably 75 mg for use as an adrenal stabilizer;
A natural source of amino acid L-Carnitine as a fat mobilizer selected from the range of 80 mg to 120 mg, and preferably 100 mg;
A natural source of saticylic acid in the form of white willow bark for encouraging thermogenesis selected from the range of 20 mg to 30 mg and preferably 25 mg;
A natural source of iodine in the form such as extracted from sea vegetables as a thyroid moderator selected from the range of 90 mcg to 110 mcg, and preferably 100 mcg;
An amount of Vitamin B6 selected from the range of 7 mg to 15 mg, and preferably 10 mg, for use as an enzyme co-factor; and
An amount of chromium polynicotinate selected from the range of 40 mcg to 65 mcg, and preferably 50 mcg, for use in balancing blood sugar levels.

7. The composition as recited in claim 1, and further comprising:
A weight of nutritional fiber agent selected from the range of 600 mg to 1200 mg and preferably 800 mg for managing insulin levels in the bloodstream, and for slowing the rate at which sugars and fats from the digestion process of the living mammal enter the bloodstream; and
Said nutritional fiber agent being selected from one or more of psyillium husks, of guar gum, of oat fiber, and of apple pectin.

8. The composition as recited in claim 2, and further comprising:
A weight of nutritional fiber agent selected from the range of 600 mg to 1200 mg, and preferably 800 mg of any one or combination of psyillium husks, guar gum, oat fiber, and apple pectin.

9. The composition as recited in claim 3, and further comprising:
A nutritional fiber agent for managing insulin levels in the bloodstream, and for slowing the rate at which sugars and fats from the digestion process of the living mammal enter the bloodstream;
said nutritional fiber agent further comprising a weight of psyillium husks selected from the range of 180 mg to 220 mg, and preferably 200 mg;
a weight of guar gum selected from the range of 7 mg to 12 mg, and preferably 10 mg;
a weight of oat fiber selected from the range of 7 mg to 12 mg, and preferably 10 mg; and
a weight of apple pectin selected from the range of 7 mg to 12 mg, and preferably 10 mg.

10. The composition as recited in claim 4, and further comprising:
A nutritional fiber agent for managing insulin levels in the bloodstream, and for slowing the rate at which sugars and fats from the digestion process of the living mammal enter the bloodstream;
said nutritional fiber agent further comprising a weight of psyillium husks selected from the range of 180 mg to 220 mg, and preferably 200 mg;
a weight of guar gum selected from the range of 7 mg to 12 mg, and preferably 10 mg;
a weight of oat fiber selected from the range of 7 mg to 12 mg, and preferably 10 mg; and
a weight of apple pectin selected from the range of 7 mg to 12 mg, and preferably 10 mg.

11. The composition as recited in claim 5, and further comprising:
a nutritional fiber agent for managing insulin levels in the bloodstream, and for slowing the rate at which sugars and fats from the digestion process of the living mammal enter the bloodstream;
said nutritional fiber agent further comprising a weight of psyillium husks selected from the range of 180 mg to 220 mg, and preferably 200 mg;
a weight of guar gum selected from the range of 7 mg to 12 mg, and preferably 10 mg;
a weight of oat fiber selected from the range of 7 mg to 12 mg, and preferably 10 mg; and
a weight of apple pectin selected from the range of 7 mg to 12 mg, and preferably 10 mg.

12. The composition as recited in claim 6, and further comprising:
a nutritional fiber agent for managing insulin levels in the bloodstream, and for slowing the rate at which sugars and fats from the digestion process of the living mammal enter the bloodstream;
said nutritional fiber agent further comprising a weight of psyillium husks selected from the range of 180 mg to 220 mg, and preferably 200 mg;
a weight of guar gum selected from the range of 7 mg to 12 mg, and preferably 10 mg;
a weight of oat fiber selected from the range of 7 mg to 12 mg, and preferably 10 mg; and
a weight of apple pectin selected from the range of 7 mg to 12 mg, and preferably 10 mg.

13. The composition as recited in claim 6, and further comprising:
A weight of nutritional fiber agent selected from the range of 600 mg to 1200 mg, and preferably 800 mg of any one or combination of psyillium husks, guar gum, oat fiber, and apple pectin.

14. The composition as recited in claim 5, and further comprising;
A weight of nutritional fiber agent selected from the range of 880 mg to 950 mg and preferably 925 mg for managing insulin levels in the bloodstream, and for slowing the rate at which sugars and fats from the digestion process of the living mammal enter the bloodstream; and
Said nutritional fiber agent being selected from one or more of psyillium husks, of guar gum, of oat fiber, and of apple pectin.

15. The composition as recited in claim 6, and further comprising:
A weight of nutritional fiber agent selected from the range of 880 mg to 950 mg and preferably 925 mg for managing insulin levels in the bloodstream, and for slowing the rate at which sugars and fats from the digestion process of the living mammal enter the bloodstream; and
Said nutritional fiber agent being selected from one or more of psyillium husks, of guar gum, of oat fiber, and of apple pectin.

16. The composition as recited in claim 4, and further comprising:
A natural source of Caffeine selected from one or more of guarana and ekola nut selected from the range of 75 mg to 125 mg, and preferably 100 mg of guarana and from a range of 40 mg, to 70 mg, and preferably 50 mg of ekola nut;
A natural source of ephedrine, rated as 8% ephedrine, in the form of Ma Huang selected from the range of 125 mg to 175 mg and preferably 150 mg;
A natural source of Siberian ginseng selected from the range of 60 mg to 80 mg, and preferably 75 mg as an adrenal stabilizer;
A natural source of amino acid L-Carnitine as a fat mobilizer selected from the range of 80 mg to 120 mg, and preferably 100 mgs;
A natural source of saticylic acid in the form of white willow bark for encouraging thermogenesis selected from the range of 20 mg to 30 mg and preferably 25 mg;
A natural source of iodine in the form such as extracted from sea vegetables as a thyroid moderator selected from the range of 90 mcg to 110 mcg, and preferably 100 mcg;
An amount of Vitamin B6 selected from the range of 7 mg to 15 mg, and preferably 10 mg, for use as an enzyme co-factor;
An amount of chromium polynicotinate selected from the range of 40 mcg to 65 mcg, and preferably 50 mcg, for use in balancing blood sugar levels; and
An amount of Kava Kava for partially neutralizing the stimulating effects of ephedrine and caffeine selected from the range of 45 mg to 55 mg, and preferably 50 mgs.

17. The composition as recited in claim 4, and further comprising:
A weight of DL-Phenylalanine selected from the range of 150 mg to 190 mg and preferably 175 mg for conversion by the living mammal into norepinephrine, dopamine, and epinephrine for use as neurotransmitters, and for use as affecting energy, hunger, and mood in a positive direction, and for extending the life of endorphins in the brain;
Said DL-Phenylalanine being converted by the living mammal into phenylethylamine as a positive mood enhancer for the living mammal;
A natural source of Caffeine selected from one or more of guarana and cola nut selected from the range of 75 mgs to 125 mgs, and preferably 100 mgs;
A natural source of Siberian ginseng for use as an adrenal stabilizer selected from the range of 120 mg to 160 mg and preferably 200 mg;
A natural source of amino acid L-Carnitine as a fat mobilizer selected from the range of 80 mgs to 120 mgs, and preferably 100 mgs;
A natural source of saticylic acid in the form of white willow bark for encouraging thermogenesis and selected from the range of 40 mg to 60 mg and preferably 50 mg;
A natural source of iodine in the form such as extracted from sea vegetables as a thyroid moderator selected from the range of 90 mcg to 110 mcg, and preferably 100 mcg;
An amount of Vitamin B6 selected from the range of 7 mgs to 15 mgs, and preferably 10 mgs, for use as an enzyme co-factor; and
An amount of chromium polynicotinate selected from the range of 40 mcg to 65 mcg, and preferably 50 mcg, for use in balancing blood sugar levels.

18. The composition as recited in claim 16, and further comprising:
a nutritional fiber agent for managing insulin levels in the bloodstream, and for slowing the rate at which sugars and fats from the digestion process of the living mammal enter the bloodstream;
said nutritional fiber agent further comprising a weight of psyillium husks selected from the range of 180 mg to 220 mg, and preferably 200 mg;
a weight of guar gum selected from the range of 7 mg to 12 mg, and preferably 10 mg;
a weight of oat fiber selected from the range of 7 mg to 12 mg, and preferably 10 mg; and
a weight of apple pectin selected from the range of 7 mg to 12 mg, and preferably 10 mg.

19. The composition as recited in claim 17, and further comprising:
a nutritional fiber agent for managing insulin levels in the bloodstream, and for slowing the rate at which sugars and fats from the digestion process of the living mammal enter the bloodstream;
said nutritional fiber agent further comprising a weight of psyillium husks selected from the range of 180 mg to 220 mg, and preferably 200 mg;
a weight of guar gum selected from the range of 7 mg to 12 mg, and preferably 10 mg;
a weight of oat fiber selected from the range of 7 mg to 12 mg, and preferably 10 mg; and
a weight of apple pectin selected from the range of 7 mg to 12 mg, and preferably 10 mg.
